(19) United States
(12) Patent Application Publication
(10) Pub. No.: US 2018/0185485 A1
(43) Pub. Date: Jul. 5, 2018

(54) Polymeric Nanoparticles for Enhancing HIFU-Induced Ablation

(71) Applicant: STICHTING KATHOLIEKE UNIVERSITEIT, Nijmegen (NL)

(72) Inventors: Mangala SRINIVAS, Utrecht (NL); Carl Gustav FIGDOR, 's Hertogenbosch (NL); Ingrid Jolanda Monique DE VRIES, Nijmegen (NL)

(21) Appl. No.: 15/740,194

(22) PCT Filed: Jul. 1, 2016
(86) PCT No.: PCT/EP2016/065600

§ 371 Date: Dec. 27, 2017

(30) Foreign Application Priority Data
Jul. 1, 2015 (EP) 15174914.0

(51) Int. Cl. A61K 41/00; A61K 49/22; A61N 7/02

(52) U.S. Cl. CPC A61K 41/0028; A61N 7/02; A61K 49/222

(57) ABSTRACT

In the field of medical therapy, more in particular in the field of ablation therapy using ultrasound, such as high intensity focused ultrasound (HIFU), devices and methods are disclosed for enhancing the ablation effect of HIFU. More in particular, a polymeric particle is disclosed, including a polymer entrapping a liquid perfluorocarbon for use in high frequency ultrasound (HIFU) ablation therapy in a human or animal body, wherein the HIFU is focused in a focal region, wherein the ablation effect of the HIFU in the focal region is enhanced by administering the particles to the human or animal body, and the liquid perfluorocarbon does not undergo a phase change from liquid to gas during exposure to the HIFU.

### FIELD OF THE INVENTION

The invention is in the field of medical therapy, more in particular in the field of ablation therapy using ultrasound, such as high intensity focused ultrasound (HIFU). The invention provides means and methods for enhancing the ablation effect of HIFU.

### BACKGROUND OF THE INVENTION

The use of ultrasound in medical imaging procedures is well known in the art. It is the most frequently used clinical imaging modality. Ultrasound is known as an economical, non-invasive, real time technique with a well-established safety record. It can be used for longitudinal studies and repeated use is not harmful for the body.

Ultrasound devices do not produce any ionizing radiation and their operation does not involve the use of radiolabels. The devices for performing ultrasound imaging are portable and already in widespread use. Ultrasound imaging is potentially quantitative and it is not a whole body imaging modality, and is therefore limited to target organs. Ultrasound imaging is limited with respect to depth of imaging.

Typically, gas-filled microbubbles are employed as contrast agents in ultrasound imaging. They commonly have a relatively large size (1000-10000 nm diameter) which is generally unsuitable for applications such as cell labeling. Moreover, they are also unsuitable for imaging outside the blood stream e.g. in tumor imaging. Such gas-filled microbubbles have a short lifetime, typically in the order of seconds to minutes. They also suffer from the additional disadvantage that cell damage, including to blood vessels, may occur as the gas bubbles burst. Moreover, gas-filled microbubbles can be unstable so that they cannot be stored for a significant amount of time; they typically have to be used soon after hydration. Finally, such large agents cannot leave the circulation and thus present very limited opportunities for in vivo targeting or drug delivery applications. Their large size also encourages prompt clearance by the kidneys, which further limits their useful lifetime in vivo.

Ultrasound contrast agents and their use are reviewed in Ultrasound contrast agents: basic principles. Eur J Radial. 1998 May; 27 Suppl 2:S157-60 and Kiessling et al., Theranostics 2011, volume 1, 127-134.

High-intensity focused ultrasound (HIFU) is a relatively new modality of therapy, in particular for use in cancer therapy. It makes use of the thermal and/or mechanical effects of ultrasound (US) to ablate tumors. The use of ultrasound energy makes that the technique is non-invasive and can be focused in a small region inside the body for a transducer of megahertz frequencies. The local temperature increases very quickly to a level (usually more than 50 degrees Celsius, such as 65 degrees Celsius), at which cell death occurs. The fast temperature drop below 43° C. outside the focal region results in little or no thermal damage in the intervening tissue between the transducer surface and focus [Tung et al., Ultrasound in Med. And Biol. 32:1103-1110 (2006)].

Surgical incision is not necessary for treating a deep tumor with HIFU and, thus, HIFU is generally considered as a noninvasive treatment modality. Moreover, HIFU does not involve radioactivity and so can be administered repeatedly. However, some remaining problems need to be addressed before HIFU can be used extensively in clinical practice. Major disadvantages of HIFU ablation are the long treatment durations, need for repeat sessions, small lesion size and difficulties in precise focusing of the treatment. Anesthesia is usually necessary, which increases the risks to patients. Therefore, an efficient method for enlarging the formed lesion (or increasing efficiency of treatment, particularly the rise in temperature) and the precision of the focal zone, thereby reducing the treatment duration needs to be developed.

In general, increasing the US intensity or the ablation duration will enlarge the lesion, but these methods may also overheat the surrounding normal tissue. Previous research shows that the presence of gas-filled bubbles near the HIFU focus can result in a larger lesion, often with greater temperature rises [Fujishiro et al. Int. J. Hypertherm. 14: 495-502 (1998); Yu et al. Urol. Res. 32: 14-19 (2004) and Kaneko et al. Eur. Radial. 15: 1415-1420 (2005)].

The administration of contrast gas-filled microbubbles can effectively reduce the treatment time or the required US intensity [Tran et al., IEEE Trans. Ultrason. Ferroelectr. Freq. Control 50: 1296-1304 (2003)]. It has also been shown that the administration of gas-filled ultrasound contrast agents can effectively increase the size of HIFU lesions and reduce the power required to form a lesion of a certain size by 30% (Tung et al, supra). However, it was also observed in that same study that the use of ultrasound contrast agents moved the greatest heating position away from the transducer focus by as much as 2 centimeters. It was concluded that gas-filled ultrasound contrast agents can effectively increase the size of the HIFU lesions, but lesion shift should be carefully considered. This is in addition to the inherent problems of precise focusing in ablation due to, for e.g. tissue deformation from breathing or physiological motion.

Summarizing, advanced tumors are often inoperable due to their size and proximity to critical vascular structures. High intensity focused ultrasound (HIFU) has been developed to non-invasively thermally ablate inoperable solid tumors. However, the clinical feasibility of HIFU ablation therapy has been limited by the long treatment times (in the order of several hours) and high acoustic intensities required.

It is known that HIFU-mediated heating may be enhanced by generating broadband acoustic emissions that increase tissue absorption and accelerate HIFU-induced heating. Unfortunately, this often requires high intensities and can be unpredictable.

### SUMMARY OF THE INVENTION

Contrary to a prejudice in the art, we have found that particles comprising a liquid perfluorocarbon that does not undergo phase change under the influence of high intensity focused ultrasound (HIFU), are very effective in enhancing the ablative effect of HIFU treatment. The invention therefore relates to a polymeric particle comprising a polymer entrapping a liquid perfluorocarbon for use in high frequency ultrasound (HIFU) ablation therapy in a human or animal body, wherein the HIFU is focused in a focal region, wherein the ablation effect of the HIFU in the focal region is enhanced by administering the particles to the human or animal body, characterized in that the liquid perfluorocarbon does not undergo a phase change from liquid to gas during exposure to the HIFU.

### DETAILED DESCRIPTION OF THE INVENTION

High intensity focused ultrasound has been one of the most effective minimally invasive techniques for localized tumor treatment, which receives extensive interest among biomedical scientists [Crouzet, S. et al., Eur. Urol.; 65: 907-914, (2014)]. By focusing the ultrasound from in vitro transducer into tumor tissues, obvious coagulative necrosis at tumor tissues can be generated due to the generation of high temperature within a few seconds [Acher, P. et al., BJU Int. 99: 28-32 (2007)]. However, traditional HIFU therapy is still not satisfactory in the therapeutic efficacy because of inevitable depth dependent decline of ultrasound energy along ultrasound pathway. Recently, a series of HIFU enhancing agents, consisting of a lipid emulsion or alternatively, of particles with a polymeric shell and fluorocarbon liquid core, have been shown to significantly enhance the HIFU therapeutic efficacy [Wang, X. et al., Small 10: 1403-1411 (2013), Niu, D. et al., Adv. Mater. 25: 2686-2692, (2013), Ming-hua, Y., et al., Biomaterials 35: 8197-8205 (2014)]. However, these particles typically undergo drastic structural changes during and after HIFU.

These particles were designed in such a way that the liquid content of the particles or emulsions underwent a phase change from liquid to gas when subjected to HIFU.

Thus, the hitherto described HIFU ablation-enhancing agents always contain a gas, or a liquid that may be converted into a gas by ultrasound treatment. These particles are thought to be effective either through a phase change from liquid to gas when subjected to ultrasound or from the initial gaseous component.

We have now found that particles comprising a liquid perfluorocarbon entrapped in a polymer, wherein the liquid perfluorocarbon does not undergo a phase change to a gas during exposure to HIFU, is at least as efficient and effective in enhancing HIFU ablation therapy. Hence the invention relates to a polymeric particle comprising a polymer entrapping a liquid perfluorocarbon for use in high frequency ultrasound (HIFU) ablation therapy in a human or animal body, wherein the HIFU is focused in a focal region, wherein the ablation effect of the HIFU in the focal region is enhanced by administering the particles to the human or animal body, characterized in that the liquid perfluorocarbon does not undergo a phase change from liquid to gas during exposure to the HIFU.

In other words, the invention relates to a method of enhancing HIFU ablation therapy, wherein the ablation effect of the HIFU is enhanced by administering particles to the human or animal body, wherein the particles comprise a liquid perfluorocarbon that does not undergo a phase change from liquid to gas during exposure to the HIFU.

Such particles have the particular advantage that they survive the HIFU treatment and can thus be used for imaging after HIFU treatment, in addition to increasing the efficiency and efficacy of the treatment. If the polymer is biodegradable, the method may also be used for drug delivery.

The term “particle” is herein understood to mean a matter which is solid when dry at room temperature and which can be recovered from a sol (a dispersion of solid dispersed in a liquid continuous phase) by precipitation and lyophilization. The particles according to the invention are also stable to repeated freeze/thaw and lyophilization cycles.

Liposomes, micelles and emulsion droplets are thus not included in the term “particles” as used herein. They consist of a liquid surfactant coating (typically a lipid) over the dispersed phase, which is also a liquid for imaging applications, except in the case of microbubbles where the dispersed phase is a gas.

Hence, “perfluorocarbon nanoparticles” mentioned in publications such as Invest Radial. 2006 March; 41 (3):305-12, Radiology. 2013 August; 268(2):470-80 are “perfluorocarbon emulsion droplets” and are not “particles” as used here. Emulsion droplets cannot be recovered intact by lyophilization, and emulsions are subject to flocculation, creaming, coalescence and/or Ostwald ripening. These effects do not apply to particles as used herein.

The term “particles” as used herein is equivalent to the term “beads” and may be used interchangeably.

In an experimental setting wherein we used a phantom with and without such particles, we observed that the particles clearly enhanced the HIFU effect both in terms of temperature increase and size of ablated region (examples 1 and 6, FIGS. 1 and 2). The experimental details are disclosed in the examples.

The phrase “enhancing HIFU ablation therapy” relates to an increase of the efficiency or effectivity in the ablative treatment with HIFU in comparison with the same treatment without any particles. Such an increase in efficiency or effectivity may be determined by a number of parameters. For instance, the particles as described herein may enhance the ablation effect by reducing the power for HIFU-mediated ablation required for obtaining a certain effect. The phrase may also refer to an increase in the peak temperature of the tissue in the focal region of the HIFU, as compared to the temperature obtained without the particles. The enhancement may also be expressed as the increase in volume of ablated tissue when particles as described herein are used.

Administering the particles may be done in a conventional way, such that the particles can reach the tumor or tissue that is to be subjected to ablation therapy. For instance, administration may be oral, intravenous or directly injected into the tumor.

The phrase “a liquid perfluorocarbon that does not undergo a phase change from liquid to gas during exposure to the HIFU” is meant to refer to a liquid perfluorocarbon that remains in the liquid phase when subjected to HIFU during and after the ablation treatment, preferably in the focal region.

In a preferred embodiment, the particles comprise a polymer selected from the group consisting of poly(lactic-co-glycolic) acid (PLGA poly(lactic acid) (PLA), poly(glycolic acid) (PGA), Polydimethylsiloxane (PDMS), or their copolymers. Such particles were found particularly suited because of their stability, and prolonged half-life. The liquid content of such particles survived the HIFU treatment without transition to the gas phase, therefore the particles may be used for imaging or for drug delivery after HIFU.

The particles for use in the present invention may range in size from millimeters to nanoscale, the use of nanoparticles however is preferred. Such particles may enter the tumor making use of the enhanced permeation and retention (EPR) effect. Preferred particles for use according to the invention have an average diameter of between 100 and 300 nanometer, preferably between 100 and 250 nanometer, such as 200 nanometer.

Solid tumors spontaneously accumulate biocompatible polymers, polymer micelles, liposomes, and nanoparticles of the 200 nm range size due to leaky nature of the newly formed tumor neovasculature and poor or missing lymphatic drainage in the solid tumor tissue. The nanoparticle size is thus the targeting mechanism here. This so-called enhanced permeation and retention (EPR) effect is relatively universal for many solid tumors and allows nanoparticles to be concentrated more than one order of magnitude compared to the surrounding tissue. For the passive, size-based accumulation (i.e., based on EPR effect) the goal of polymer coating is to make the particles nonimmunogenic and stealthy for the reticulo-endothelial system in order not to be scavenged before reaching tumor tissue. The nanoparticles are therefore preferably coated with polymers with known biocompatibility, i.e. poly(ethylene oxide), poly(2-alkyl-2-oxazolines) or poly[N-(2-hydroxypropyl)methacrylamide]. The polymers may be anchored to nanoparticle surface via copolymerized cholesteryl groups which have high affinity to surfaces of hydrophobic polyesters such as PLGA, PLA or PGA.

To enhance accumulation in denser or less vascularised tumors, particles may be actively targeted, for instance by using cyclic RGD peptide. This peptide is known to be selective to the integrin-expressing tumor neovasculature, which is readily available for the nanoparticles circulating in the bloodstream. Other strategies may involve antibodies, such as human or humanized antibodies, monoclonal antibodies or the like against surface tumor characteristics, which have been already validated in clinic to bind and to distribute into the tumor tissue (trastuzumab, bevacizumab, cetuximab, pertuzumab, rituximab, etc.), or other tumor targeting agents.

In a preferred embodiment of the invention, the particle for use is a nano-particle.

The intensity of the HIFU very much depends on the size, nature, composition and density of the particles. Preferably, the intensity of the high frequency ultrasound in the focal region is between 1 and 10,000 Watt.

The polymeric particles for use in the invention comprise a liquid perfluorocarbon. Preferred perfluorocarbons include perfluoropolyethers, perfluoro crown ethers, perfluorooctane and perfluorooctylbromide. The liquid perfluorocarbon is preferably a perfluoro crown ether, such as a perfluoro crown ether selected from the group consisting of perfluoro-15-crown-5-ether, perfluoro-12-crown-4-ether and perfluoro-18-crown-6-ether. The term “perfluoro crown ether” (PFCE) is to be interpreted as a cyclic perfluorocarbon containing carbon, oxygen and fluorine covalently bound in a stable ring structure.

A particularly useful perfluoro crown ether is perfluoro-15-crown-5-ether the structure of which is shown in Formula 1.

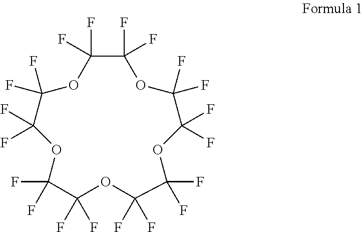 [SMILES: FC1(F)OC(F)(F)C(F)(F)OC(F)(F)C(F)(F)OC(F)(F)C(F)(F)OC(F)(F)C(F)(F)OC1(F)F]

In a further preferred embodiment, the particles for use according to the invention additionally comprise a metal chelate, such as a rare earth metal chelate, such as gadolinium chelate. In a further preferred embodiment, the gadolinium chelate is gadoteridol. The structure of gadoteridol is shown in Formula 2.

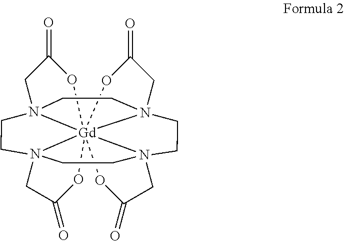

In a further preferred embodiment, the particles may comprise a detecting agent, such as a dye, such as a fluorescent dye, iodine, a carbon/graphene/quantum dot or a radionuclide. They may also comprise a therapeutic agent or a targeting agent, such as a drug, a receptor ligand or an antibody.

In a further preferred embodiment, the particles for use in the invention comprise an agent for enhancing radiotherapy, such as a metal particle, such as a heavy metal particle, such as an iron oxide or bismuth compound.

In a further preferred embodiment, the particles for use in the invention are essentially surfactant free or surfactant free.

### BRIEF DESCRIPTION OF THE DRAWINGS

FIG. 1: Temperature mapping data of a tissue phantom without (control) and with particles. The color scale shows the temperature in degrees Celsius.

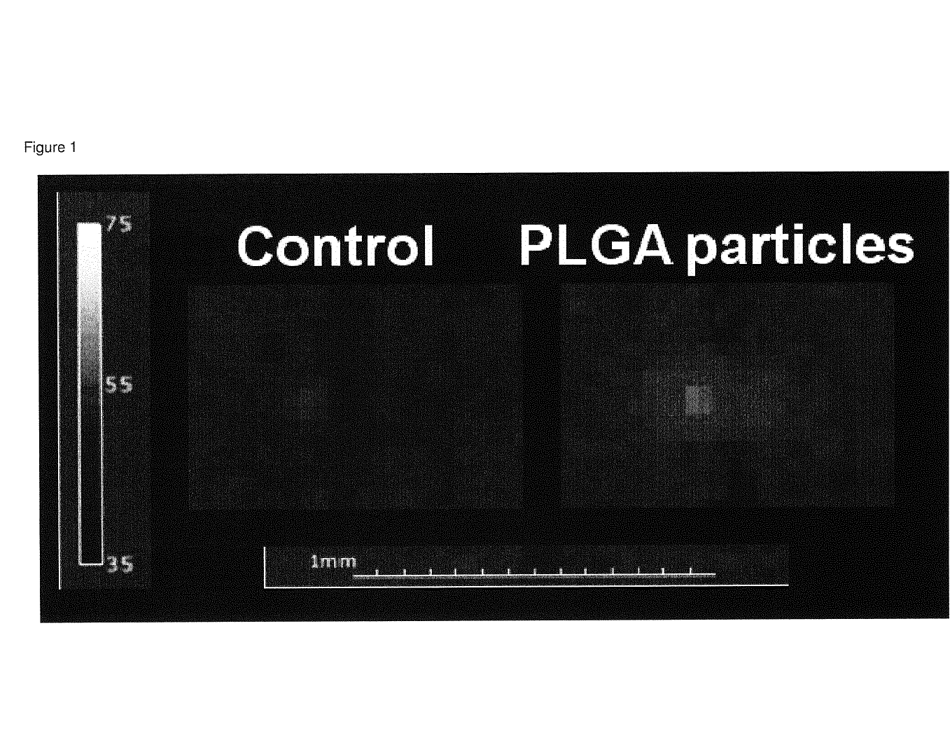

FIG. 2: Five mg of PLGA-PFCE-Gd particles were injected in tissue ex vivo, followed by 50 Watt HIFU ablation. Areas with and without nanoparticles are circled. The ablation size is clearly enhanced by the nanoparticles.

FIG. 3: Gel phantoms containing the particles indicated in Table 4. Particles were dispersed homogeneously in the gel. MR temperature mapping shows that the particles comprising PFCE (with and without Gd) enhanced heating to a value far above empty particles (PLGA only, no perfluorocarbon) or empty gel. The temperature changes are shown in table 5. Empty gel=no particles; PLGA NPs=PLGA nanoparticles with no perfluorocarbon; PLGA-PFCE-Gd NPs=PLGA nanoparticles with perfluoro-crown ether and gadolinium; PLGA-PFCE NPs=PLGA nanoparticles with perfluoro-crown ether.

### EXAMPLES

### Example 1: HIFU Enhancement in a Tissue Model with Polymeric Particles

Particles prepared according to example 2 with a high gadolinium content were injected in a sample of chicken breast that served as a tissue phantom (10 mg/ml). HIFU was carried out at 38 W with a 2 second pulse on a Bruker Clinscan system (7 T horizontal bore). The relevant tissue was then sectioned to directly visualize the ablated zone. Temperature changes were also measured in real time using standard MR thermometry sequences. Comparable results were obtained with the same particles comprising medium and low content of gadolinium. Particles without the gadolinium also showed an enhancement of the ablation effect, although this was less than particles with the low gadolinium content.

### Example 2: Production of Nanoparticles

PLGA (0.09 gram) was dissolved in 3 ml dichloromethane in a glass tube. Liquid perfluoro-15-crown-5-ether (890 microliter) was added followed by 50 ml of a solution of Prohance (a 3 mg/ml solution of Gadoteridol) diluted in water. Optionally, additional agents, such as a fluorescent dye, may be added to the fluorocarbon at this stage. If a fluorescent particle was required, 1 mg of IcG or IC-Green (Indocyanine Green, Akorn Pharmaceuticals) was added to the solution.

We prepared particles with a high, medium and low content of gadolinium. For that purpose, the above mentioned solution of Prohance® in water comprised 11.5, 5.75 and 2.85 ml respectively of Prohance® added up with water to 50 ml of solution. The entire mixture was then added dropwise into 25 ml of a solution of polyvinyl alcohol in water (20 gram/liter) under constant sonication (Branson Digital Sonifier 250; 3 minute cycle with 60 sec on and 10 sec off and maximum temperature of 20 degrees Celsius and amplitude of 30%; a cuphorn was used) The resulting emulsion was then placed at 4 degrees Celsius and allowed to evaporate with constant stirring for about 12 hours until 24 ml of solution remained. An equal volume of water was then added and the emulsion was centrifuged at 21000 g for 30 minutes at 4 degrees Celsius. The pellet was washed with water twice and the resultant suspension was lyophilized at −60 degrees Celsius, for at least 24 hours, The particles were then placed in sealed tubes and stored at −80 deg Celcius. Unless stated otherwise, the particles used in the experiments described herein are the particles with the highest gadolinium content.

### Example 3: Characterisation of Particles

We found that particles as prepared above were stable for at least a year when kept at −20 degrees Celsius in the dry form. The particles were also stable in solution at working concentrations for at least 3 months at minus 4 degrees Celsius.

Diameter of particles prepared according to example 2 was determined using dynamic light scattering (DLS) as previously described (Biomaterials. 2010 September; 31(27):7070-7). The particle size ranged from 80 to 500 nm with a sharp peak at 181 nm.

The particle diameter distribution remained stable for several months. The particles were lyophilised and frozen for storage. However, particles stored as aliquots in water (frozen) were also stable.

The particles prepared according to example 2 with high and medium gadolinium content, dissolved in water at a concentration of 1 mg/ml appeared to be exceptionally stable under conditions of ultrasound imaging. We measured particle diameter and count rate (indicative of number of particles) before and after exposure to HIFU and low and high intensity ultrasound MI (MI=0.1 and 2.0) for 30 sec. We found that the particles were not destroyed by HIFU or normal ultrasound. We also observed that increasing Gd content improves stability of the particles to ultrasound exposure. We found no change in the diameter, count rate or polydispersity (POI, indicative of the spread of diameter distribution) after exposure to high energy ultrasound for 30 sec.

It is concluded from the data that the particles as described herein are stable under even the harshest ultrasound conditions and that increasing Gd content improves stability of the particles to ultrasound exposure.

### Example 4: Gadolinium Improves Imaging Properties of the Particles

PIGNPFCE particles were prepared according to example 2 with Gd and tested for ultrasound and MRI (including 1H MRI) visibility. It was found that the addition of gadolinium enhances MRI signal (1H) and can also enhance ultrasound visibility. It is concluded that the addition of gadolinium provides an improvement of the visibility of particles comprising a fluorinated organic compound. Therefore, the particles may be visualized by using normal ultrasound or MRI (both 1H and 19F) after ablation treatment with HIFU, and this visibility may be further enhanced by adding Gd.

### Example 5: Alternative Synthesis of Particles

Particles were made as described previously [Srinivas, M. et al. Customizable, multi-functional fluorocarbon nanoparticles for quantitative in vivo imaging using 19F MRI and optical imaging. Biomaterials 31, 7070-7077 (2010)], with the addition of gadoteridol from ProHance (Bracco Imaging Europe, Amsterdam). Briefly, 1 g polyvinyl alcohol dissolved in 50 ml water only or water and, optionally, ProHance, 1780 μl, is added dropwise to 180 mg of PIGA (Resomer RG 502 H, lactide: glycolide molar ratio 48:52 to 52:48; Boehringer Ingelheim, Germany) dissolved in dichloromethane with 890μ1 PFCE (Exfluor Inc, Texas USA), or alternatively 232μ1 PFO (Perfluoron, Alcon Inc), on ice, with sonication using a Digital Sonifier 250 (Branson, Danbury, USA) with a cuphorn running at 40% power for 2 minutes in 10 second pulses. Dynamic light scattering was done on a Malvern Zetasizer Nano. Gd content was measured using mass spectrometry.

### Example 6: Further Alternative Synthesis of Particles

PIGA (100 mg, resomer 502H) was dissolved in 3 ml dichloromethane. Perfluoro-15-crown-5 ether (900 μl) and Prohance (1.78 ml) were added to the solution of PIGA and a first emulsion was formed by sonication using a microtip having a tip diameter of 3 mm at an amplitude of 40% for 15 seconds (Digital Sonifier s250 from Branson). This first emulsion was rapidly (within 10 seconds) added to a solution of poly(vinyl alcohol) (25 g of water and 100-500 mg of PVA) in a round bottom flask while sonication of PVA-containing flask was started. The entire mixture was sonicated in ice-water bath using a microtip having a tip diameter of 3 mm at an amplitude of 20% or 40% to obtain a second emulsion. The duration of the period from the addition of the first emulsion to the end of the sonication was 3 minutes (Digital Sonifier s250 from Branson).

After sonication dichloromethane was evaporated at 4° C. or room temperature overnight under stirring to achieve solidification of the beads. The beads were isolated by centrifugation at 27200 g for 35 min in 50 ml centrifugation tubes and resuspended in 25 g of water. The washing step was repeated two more times with resuspention by sonication after second washing (sonication bath, Diagenode Bioruptor). After washing, beads were resuspended in 4 ml of water, frozen with liquid N2 and freeze-dried. The resulting product was a white powder.

The amounts of the components and the sonication amplitude which were varied are shown in Table 1, together with the properties and the yield of the beads.

TABLE 1

| | | | Radius | | | |
|---|---|---|---|---|---|---|
| | | | (DLS; | | PFCE- | |
| Exp. | PVA/ | Sonication | intensity)/ | | content/ | |
| No | mg | Amplitude | nm | PDI | wt.-% | yield/mg |
| 1 | 100 | 20% | 357 | 0.49 | 11 | 77 |
| 2 | 500 | 20% | 121 | 0.1 | 5.3 | 55 |
| 3 | 100 | 40% | 314 | 0.39 | 28 | 137 |
| 4 | 200 | 40% | 174 | 0.2 | 34 | 189 |
| 5 | 350 | 40% | 146 | 0.15 | 39 | 184 |
| 6 | 500 | 40% | 121 | 0.123 | 45 | 204 |

Small beads with narrow particle size distribution were obtained by the process according to the invention (Experiments 5 and 6). It can be observed that a high amplitude (40%) and a large amount of PVA (350 mg or 500 mg) resulted in a desirable combination of a small radius, low PDI, a high PFCE content and a high yield.

### Example 7: Preparation of Beads without a Metal Compound

PLGA (100 mg, resomer 502H) was dissolved in 3 ml dichloromethane (DCM) followed by addition of perfluoro-15-crown-5 ether (900 μl). The resulting double phase liquid was rapidly added with a glass pipette to solution of poly(vinyl alcohol) (25 g of water and 100-500 mg of PVA) in a round bottom flask while sonication was started. Care was taken so that the phase of PLGA/DCM and the phase of PFCE were added simultaneously at a constant ratio. The entire mixture was sonicated in ice-water bath using a microtip having a tip diameter of 3 mm at an amplitude of 20% or 40% to obtain an emulsion. The duration of the period from the addition of the double phase liquid to the end of the sonication was 3 minutes (Digital Sonifier s250 from Branson).

After sonication dichloromethane was evaporated at 4° C. or room temperature overnight under stirring to achieve solidification of the beads. The beads were isolated by centrifugation at 27200 g for 35 min in 50 ml centrifugation tubes and resuspended in 25 g of water. The washing step was repeated two more times with resuspention by sonication after second washing (sonication bath, Diagenode Bioruptor). After washing, beads were resuspended in 4 ml of water, frozen with liquid N2 and freeze-dried. The resulting product was a white powder with a yield of at least 100 mg.

TABLE 2

| | | | Radius | | | |
|---|---|---|---|---|---|---|
| | | | (DLS; | | PFCE- | |
| Exp. | PVA/ | Sonication | intensity)/ | | content/ | |
| No | mg | Amplitude | nm | PDI | wt.-% | yield/mg |
| 7 | 100 | 20% | 354 | 0.25 | 15 | 86 |
| 8 | 100 | 40% | 339 | 0.24 | 25 | 116 |
| 9 | 500 | 40% | 100 | 0.04 | 48 | 154 |

Small beads with narrow particle size distribution and a high PFCE content were obtained with a high yield according to the process of the invention (Ex 9).

### Experiments 10-12

Experiment 6 was repeated except that the PLGA was dissolved in a solvent indicated in Table 3.

TABLE 3

| | | | | Radius | | | |
|---|---|---|---|---|---|---|---|
| | | | | (DLS; | | PFEC- | |
| Exp. | | PVA/ | Sonication | intensity)/ | | content/ | yield/ |
| No | Solvent | mg | Amplitude | nm | PDI | wt.-% | mg |
| 10 | THF | 500 | 40% | 171 | 0.5 | 15 | 131 |
| 11 | Acetone | 500 | 40% | 294 | 0.66 | 8 | 90 |
| 12 | Acetoni- | 500 | 40% | 223 | 0.22 | 5 | 95 |
| | trile | | | | | | |

Beads obtained are larger and have a broader size distribution than the experiments in which the solvent was dichloromethane.

Diameter of beads prepared according to examples 1-12 was determined using dynamic light scattering (DLS) as described in Biomaterials. 2010 September; 31 (27):7070-7.

### Example 8: Preparation of Beads Using Cup Horn (Experiment 13)

PLGA (90 mg, resomer 502H) was dissolved in 3 ml dichloromethane.

Perfluoro-15-crown-5 ether (890 μl) was added to the solution of PLGA. 50 ml of an aqueous solution comprising of Prohance with concentration of 3 mg/ml was further added. This mixture was added dropwise to a solution of poly(vinyl alcohol) (20 g/l) in a glass tube while sonication of PVA-containing flask was started. The entire mixture was sonicated in a cup horn at an amplitude of 30% for 3 minutes, with 60 s on and 10 s of cycles (Digital Sonifier s250 from Branson) to obtain a second emulsion. During the sonication the temperature of the cooling water was maintained at 4° C. by a refrigerated circulator.

After sonication dichloromethane was evaporated at 4° C. overnight under stirring to achieve solidification of the beads. The beads were isolated by centrifugation at 21000 g for 30 min in 2 ml centrifugation tubes and resuspended in 25 g of water. The pellet was washed with water twice and then resuspended in water, frozen at −80° C. and freeze-dried. The resulting product was a white powder with a yield of 50 mg.

The examples according to experiments 5, 6 and 9 resulted in a much higher yield compared to experiment 13.

### Example 9: Nanoparticles Enhance HIFU-Induced Ablation in Tissue

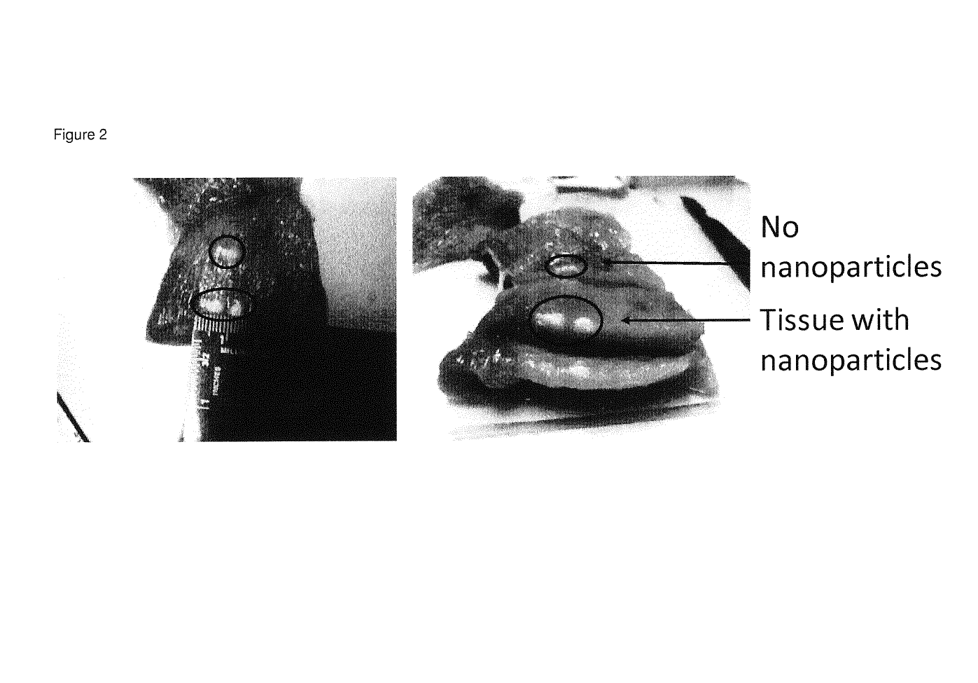

PLGA-PFCE-Gd nanoparticles as described herein were injected into chicken breast ex vivo. The sample was then subjected to HIFU ablations, using a standard in vivo setting of 50 Watt. Four ablations were carried out for each area i.e. with and without the nanoparticles. The tissue was then sliced (FIG. 2, right panel) to examine the extent of the ablated tissue. It was found that the nanoparticles clearly enhanced the ablated tissue area.

### Example 10: Nanoparticles Enhance HIFU-Induced Ablation In Vitro

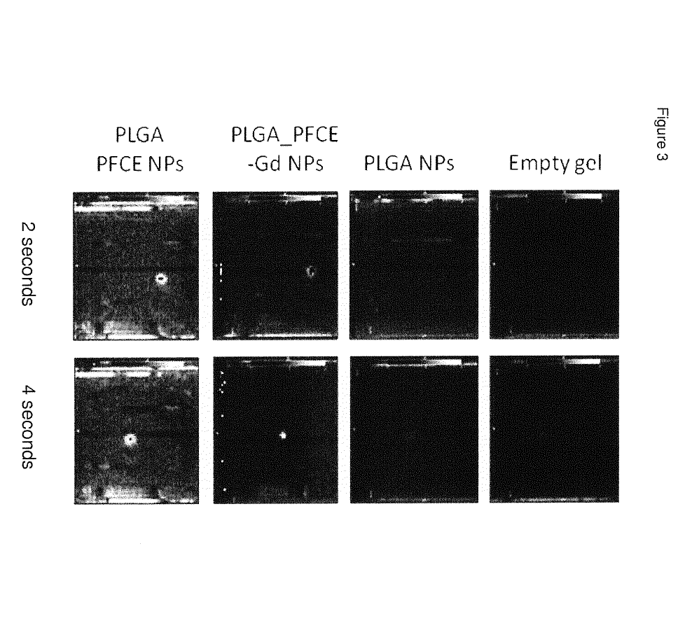

Agarose gel phantoms containing homogeneously distributed nanoparticles, or an empty control, were treated at room temperature using MRI-guided HIFU using a standard in vivo setting of 50 Watt. MR thermometry was also carried out (FIG. 3). The aim of this experiment was to measure the effect of the nanoparticles in terms of temperature changes. Table 4 summarizes the properties of the particles used, with diameter and polydispersity (PDI) measured using dynamic light scattering.

TABLE 4

| Particles | Diameter (nm) | PDI |
|---|---|---|
| Empty gel (no particles) | n/a | n/a |
| Empty PLGA | 225 ± 8 | 0.11 |
| PLGA/PFCE/Gd | 285 ± 4 | 0.13 |
| PLGA/PFCE | 180 | 0.14 |

The MRI images (FIG. 3) show that the particles clearly enhance the heating effect, well over either empty gel or empty PLGA nanoparticles without any perfluorocarbon.

Table 5 shows the peak temperatures measured in the gels of FIG. 3.

TABLE 5
Peak temperatures after 2 or 4 seconds of HIFU.

| | 2 seconds | 4 seconds |
|---|---|---|
| | ablation | ablation |
| Nanoparticles | [degrees C.] | [degrees C.] |
| No particles | 50 | 46 |
| Empty PLGA nanoparticles | 50 | 48 |
| PLGA-PFCE nanoparticles | 70 | 70 |
| PLGA-PFCE nanoparticles + Gadolinium | 71 | 75 |

It is to be noted that the temperature increase with the nanoparticles containing perfluorocarbon was very high, and this resulted in an underestimation in the peaks of the thermometry scans. It is concluded from the data shown in table 5 that the no particles control as well as the PLGA particles without perfluorocarbon (empty PLGA nanoparticles) show relatively low temperature increases, whereas the perfluorocarbon containing particles caused a much higher increase in temperature.

### Example 11: Stability of Particles Under HIFU Conditions

In this experiment we looked at the effect of high temperature on the nanoparticles, so see if the particles remain intact (i.e. without structural changes) after exposure to high temperature. For this, the particles were incubated at either 50 or 80 degrees C. for 0.5 or 2 hours. This is far in excess of the few seconds of exposure to high temperatures that occurs during HIFU.

It was found that even in these harsh conditions, no change in particle diameter or polydispersity, as measured by dynamic light scattering, was observed in all conditions (including up to 2 hours at 80 degrees Celsius). This shows that the particles are not damaged or destroyed by HIFU ablation, as the diameter does not change. It was also found that the polydispersity index (POI) never rose above 0.1, indicating a very homogeneous distribution of particle diameters. This, again, shows that the particles do not undergo any structural changes due to high temperature.

TABLE 6
Stability of PLGA/PFCE/Gd nanoparticles
at elevated temperatures.

| Time | Diameter at 50 | Diameter at 80 | PDI at 50 | PDI at 80 |
|---|---|---|---|---|
| [hours] | degrees C. [nm] | degrees C. [nm] | degrees C. | degrees C. |
| 0 | 190 | 190 | 0.07 | 0.07 |
| 0.5 | 200 | 200 | 0.07 | 0.09 |
| 2 | 200 | 200 | 0.06 | 0.07 |

What is claimed is:

1. A polymeric particle, comprising: a polymer entrapping a liquid perfluorocarbon for use in high intensity focused ultrasound (HIFU) ablation therapy in a human or animal body, wherein the HIFU is focused in a focal region, wherein an ablation effect of the HIFU in the focal region is enhanced by administering the particle to the human or animal body, wherein the liquid perfluorocarbon does not undergo a phase change from liquid to gas during exposure to the HIFU.

2. The particle according to claim 1, wherein the polymer comprises poly(lactic-co-glycolic) acid (PLGA), poly(lactic acid) (PLA), poly(glycolic acid) (PGA), Polydimethylsiloxane (PDMS), or their copolymers.

3. The particle according to claim 1, wherein the particle is a nano-particle.

4. The particle according to claim 1, wherein the intensity of the high frequency ultrasound in the focal region is between 1 and 10,000 Watt.

5. The particle according to claim 1, wherein the liquid perfluorocarbon is a perfluoro crown ether.

6. The particle according to claim 5, wherein the perfluoro crown ether is selected from the group consisting of perfluoro-15-crown-5-ether, perfluoro-12-crown-4-ether and perfluoro-18-crown-6-ether.

7. The particle according to claim 1, additionally comprising a metal chelate.

8. The particle according to claim 7, wherein the metal chelate is a rare earth metal chelate.

9. The particle according to claim 8, wherein the rare earth metal chelate is gadolinium chelate.

10. The particle according to claim 9, wherein the gadolinium chelate is gadoteridol.

11. The particle according to claim 1, wherein the particle comprises a detecting agent.

12. The particle according to claim 1, wherein the particle comprises a therapeutic agent or a targeting agent.

13. The particle according to claim 1, wherein the particle has a diameter between 100 and 300 nanometer.

14. The particle according to claim 1, which is essentially surfactant free or surfactant free.

15. The particle according to claim 11, wherein the detecting agent is a fluorescent or luminescent agent.

16. The particle according to claim 15, wherein the fluorescent or luminescent agent is a dye or a radionuclide.

17. The particle according to claim 12, wherein the therapeutic agent or targeting agent is a drug, a receptor ligand or an antibody.

18. The particle according to claim 13, wherein the particle has a diameter between 100 and 250 nanometer.

19. The particle according to claim 18, wherein the particle has a diameter of 200 nanometer.

20. A polymeric particle, comprising: a polymer entrapping a liquid perfluorocarbon for use in high intensity focused ultrasound (HIFU) ablation therapy in a human or animal body, wherein the HIFU is focused in a focal region, wherein an ablation effect of the HIFU in the focal region is enhanced by administering the particle to the human or animal body, wherein the liquid perfluorocarbon does not undergo a phase change from liquid to gas during exposure to the HIFU,
wherein the polymer comprises poly(lactic-co-glycolic) acid (PLGA), poly(lactic acid) (PLA), poly(glycolic acid) (PGA), polydimethylsiloxane (PDMS), or their copolymers,
wherein the liquid perfluorocarbon is a perfluoro crown ether,
wherein the perfluoro crown ether is selected from the group consisting of perfluoro-15-crown-5-ether, perfluoro-12-crown-4-ether and perfluoro-18-crown-6-ether.

21. The particle according to claim 20, additionally comprising a metal chelate.

22. The particle according to claim 21, wherein the metal chelate is a rare earth metal chelate.

23. The particle according to claim 22, wherein the rare earth metal chelate is gadolinium chelate.

24. The particle according to claim 23, wherein the gadolinium chelate is gadoteridol.

25. A polymeric particle, comprising: a polymer entrapping a liquid perfluorocarbon for use in high intensity focused ultrasound (HIFU) ablation therapy in a human or animal body, wherein the HIFU is focused in a focal region, wherein an ablation effect of the HIFU in the focal region is enhanced by administering the particle to the human or animal body, wherein the liquid perfluorocarbon does not undergo a phase change from liquid to gas during exposure to the HIFU,
wherein the polymer comprises poly(lactic-co-glycolic) acid (PLGA), poly(lactic acid) (PLA), poly(glycolic acid) (PGA), polydimethylsiloxane (PDMS), or their copolymers,
wherein the particle additionally comprises a metal chelate.

26. The particle according to claim 25, wherein the liquid perfluorocarbon is a perfluoro crown ether.

27. The particle according to claim 26, wherein the metal chelate is a rare earth metal chelate.
